(19) United States
(12) Patent Application Publication
(10) Pub. No.: US 2005/0249631 A1
(43) Pub. Date: Nov. 10, 2005

(54) Method and apparatus for ozone disinfection of water supply pipelines

(76) Inventors: Christopher R. Schulz, Aurora, CO (US); Stephen R. Lohman, Conifer, CO (US)

Correspondence Address: ALFRED J MANGELS, 4729 CORNELL ROAD, CINCINNATI, OH, 452412433, US

(21) Appl. No.: 11/065,768

(22) Filed: Feb. 25, 2005

Related U.S. Application Data
(63) Continuation-in-part of application No. 10/243,971, filed on Sep. 14, 2002, now abandoned.

(51) Int. Cl. A61L 2/18

(52) U.S. Cl. 422/28; 422/292

(57) ABSTRACT

A method and apparatus for disinfecting the interior of pipelines and conduits, particularly water mains. Ozone is utilized as the disinfectant vehicle to neutralize microbial contamination of the conduit. An ozone generation system includes a venturi injector for introducing ozone into pressurized water to provide a treating solution that is introduced into and that flows along a predetermined length of the conduit to be treated. The ozone concentration is regulated to maintain an ozone residual at the conduit outlet of about 0.2 mg/L to about 0.3 mg/L for a time sufficient to assure the desired level of disinfection of the conduit interior. Carbon dioxide can be added to improve the ozone residual stability and the effectiveness of disinfection. The apparatus is transportable and can be carried by a transportation vehicle, such as a truck or trailer, for on-site disinfection of pipelines at varying sites.

### CROSS-REFERENCE TO RELATED APPLICATION

This application is a continuation-in-part of copending application Ser. No. 10/243,971, filed on Sep. 14, 2002, the entire disclosure of which is hereby incorporated herein by reference.

### BACKGROUND OF THE INVENTION

1. Field of the Invention

The present invention relates to a method and apparatus for disinfection of liquid-carrying conduits. More particularly, the present invention relates to a method and apparatus for quickly disinfecting water pipelines and other conduits by the introduction into the pipelines and conduits of controlled amounts of ozone-containing water to disinfect the interior surfaces of the conduits.

2. Description of the Related Art

Microbial contamination within new or repaired water mains has been associated with several waterborne disease outbreaks in public water supply systems. Currently, chlorine is the most commonly-utilized disinfectant for treating water mains and conduits. Practices recommended by the American Water Works Association to treat the interior of water-carrying conduits include several techniques that have a number of shortcomings, including the handling and on-site preparation of hazardous chemical solutions, uncertainty of the effectiveness of the treatment, the need to carry out a dechlorination step before disposal of the chlorinated discharges, the need to dispose of large volumes of dechlorinated water, and the length of the exposure time to the chlorine that is necessary to ensure adequate disinfection.

Among the treatment methods currently utilized are the continuous feed method, the slug method, and the tablet method. In the continuous feed method the conduit is first flushed with a strong chlorine solution, and the conduit is then filled with a solution having at least 25 mg/L of free chlorine. That solution is retained within the conduit so that a residual of at least 10 mg/L is maintained after the passage of 24 hours. In the slug method a slug dose of free chlorine having a concentration greater than 100 mg/L is caused to move slowly through the conduit so that all interior surfaces are exposed to the highly concentrated chlorine solution for a period of not less than three hours. And in the tablet method calcium hypochlorite tablets are attached to the conduit inner surface at several axially spaced positions, after which the conduit is filled with water to dissolve the tablets, so that a residual of at least 25 mg/L is maintained in contact with the conduit inner surface for at least 24 hours.

Although generally effective, the methods presently employed have several drawbacks. First of all, the slug and continuous feed methods require the use, transport, and on-site preparation of hazardous hypochlorite and sodium bisulfite solutions in trailer or truck-mounted storage tanks for the chlorination and dechlorination steps. Secondly, the 24-hour minimum holding time for the slug and continuous feed methods to ensure adequate disinfection involves lengthy delays that adversely affect construction time schedules. And sometimes in the tablet method the tablets do not fully dissolve within the conduit, and because in that method the water is static, incomplete dissolving of the tablets can result in local areas of the conduits that are thereby not effectively disinfected. Furthermore, each of the chlorine-based methods requires dechlorination of the treatment solutions to allow disposal by discharge of the solutions into sanitary or storm sewers, into storage ponds, or into flood control channels.

In addition to the material handling, the disposal, and the time delay factors noted above, the conduit disinfection methods in common use today also are not linked to a scientifically rational disinfection basis. The concentration and exposure time criteria are relatively arbitrary, as contrasted with the concentration x contact time (CT) concepts that form the basis for disinfection in modern drinking water treatment systems.

It is an object of the present invention to overcome the problems and shortcomings noted above in connection with the presently-utilized conduit disinfection methods.

### SUMMARY OF THE INVENTION

Briefly stated, in accordance with one aspect of the present invention, a method is provided for disinfecting liquid-carrying conduits. The method includes providing a conduit to be disinfected, wherein the conduit includes an inlet connection and an outlet connection that are spaced from each other along the conduit to define a predetermined conduit length between the inlet connection and the outlet connection. Pressurized water from a potable water source is introduced into an ozone treatment system, and ozone is injected into the pressurized water within the ozone treatment system and at an ozone dose sufficient to maintain a predetermined ozone-in-water residual concentration at the outlet connection of the conduit to be disinfected. Pressurized ozonated water from the ozone treatment system is introduced into the conduit at the inlet connection, and a flow of the ozonated water is maintained within the conduit from the inlet connection to the outlet connection. The discharge of water from the outlet connection is regulated to maintain a predetermined water pressure and a predetermined ozone-in-water residual concentration at the outlet connection over a sufficient period of time to meet a disinfection requirement.

In accordance with another aspect of the invention, apparatus is provided for disinfecting a liquid-carrying conduit. The apparatus includes a source of pressurized water, a source of ozone, and means for introducing the ozone into the pressurized water to provide an ozone-containing disinfectant liquid. The conduit to be disinfected includes an inlet connection for introducing the ozone-containing disinfectant liquid into the conduit at a first location, and an outlet connection at a second location spaced along a conduit central axis from the first location for allowing the disinfectant liquid to flow through the conduit from the first location to the second location and to exit from the conduit. Means are provided for introducing the disinfectant liquid into the conduit at the inlet connection, and flow control means are provided for regulating the rate of flow of the disinfectant liquid within the conduit to expose the interior surfaces of the conduit to the disinfectant liquid for a time sufficient to meet predetermined disinfection requirements.

In accordance with a further aspect of the present invention, an ozone treatment system is provided for introducing ozone into water under pressure for disinfection purposes. The ozone treatment system includes a source of ozone and a source of pressurized potable water. Means are provided for introducing the ozone into the water and an analyzer is provided for determining the rate of decay of the ozone residual concentration of the water. A regulator controls the rate of ozone introduction into the water as a function of information provided by the decay rate analyzer to provide a predetermined ozone concentration in the water to meet disinfection requirements.

### BRIEF DESCRIPTION OF THE DRAWINGS

The structure, operation, and advantages of the present invention will become further apparent upon consideration of the following description, taken in conjunction with the accompanying drawings in which:

FIG. 1 is an illustrative graph of CT (concentration x contact time) accumulation rates within a water system pipeline section as a function of exposure time and pipeline section length.

FIG. 2 is a schematic view of an exemplary embodiment of a pipeline segment and of an ozone treatment system for ozone disinfection of the pipeline inner surface.

FIG. 3 is a graph showing the effect of carbon dioxide addition on ozone residual stability.

### DESCRIPTION OF THE PREFERRED EMBODIMENTS

In the present invention the disinfection of a pipeline or conduit is achieved by exposing the inner surfaces of the pipeline or conduit to a solution of treated water, such as potable water from a municipal water system, into which ozone has been dissolved. The use of ozone for disinfection eliminates the need for a post-exposure treatment of the solution, such as by a dechlorination step, because ozone decays to oxygen in water over a relatively short time period, typically less than one hour. The rate of decay depends upon the water temperature, the pH, the concentration of ozone-demanding substances in the water, including other disinfectant residuals such as chlorine or chloramines, and the carbonate alkalinity of the water.

The ozone decay to oxygen factor makes it possible to develop an ozone-based disinfection process that allows the ozone residual within the conduit to decay to oxygen before discharging the treating solution from the conduit into the environment, thereby simplifying the treating solution disposal process while simultaneously avoiding environmental harm. In fact, depending upon the ozone residual concentration of the treating solution, the water from an ozone-based disinfection process often can safely be flushed directly onto streets, or into sewers or watercourses. The low ozone residuals in the contemplated treatment solution, less than about 1 mg/L, will quickly be consumed upon contact with pavement or dirt, or upon exposure to ultraviolet light from the sun.

The use of an ozone-based treatment solution also avoids the storage, transportation, and on-site preparation of the hazardous chemicals normally involved in chlorine-based disinfection processes utilizing hypochlorites and bisulfites. In that regard, the ozone can be generated on-site through an electrical process using oxygen as the feed gas, which, in turn, is generated from an on-site oxygen separation system or is provided in pressurized 150-lb oxygen storage cylinders. The equipment needed is compact and can be used in the field without the need for large storage facilities or for large transportation vehicles.

Because ozone is one of the most powerful disinfectants for drinking water, the ozone-based conduit disinfection process can be accomplished in minutes, rather than in the hours required in the chlorine-based processes. Ozone is capable of meeting disinfection targets for protozoa, bacteria, and viruses at CT values that are around two orders of magnitude lower than those required for chlorine-based processes. Accordingly, it is possible to utilize a flow-through process that minimizes the lengthy holding times that are associated with the chlorine-based processes.

It has been known that substantial levels of heterotrophic bacteria are commonly present in both new and older tuberculated water mains. The bacterial concentration on the internal surface of the main is generally proportional to the pipeline diameter. Prior research sponsored by the American Water works Association showed that the 90^{th} percentile heterotrophic plate count (HPC) for bacterial populations on different pipeline surfaces was about 8×10⁸ coliform forming units (cfu) per square foot of pipeline surface. For 8-inch and 108-inch pipelines that value corresponds with HPC concentrations of 170,000 cfu/ml and 12,500 cfu/ml, respectively. Those research-based values were utilized to establish the HPC inactivation requirements for ozone disinfection of water mains.

In AWWA Standard C651, which applies to construction of new water mains, an HPC concentration of 500 cfu/ml is considered acceptable for water distribution systems. By utilizing a 10-fold safety factor, a final HPC value of 50 cfu/ml was selected as a more stringent target for a reliable, ozone-based disinfection process for water mains. Using the initial HPC concentrations for the two pipeline diameters referred to above, the required log inactivation to meet that target value would be 3.5 and 2.4 logs, respectively. Accordingly, a conservative water main disinfection goal of 4 log (or 99.99%) HPC inactivation is suggested for ozone disinfection of water mains. Based upon prior research, the corresponding CT product for 4-log HPC inactivation by ozone ranges from approximately 0.5 to about 5 mg/L/min, depending upon water temperature and the sensitivity of the bacterial population to ozone.

The CT product concept is utilized to measure the effectiveness of ozone for disinfecting water mains. That is similar to the approach utilized in water treatment plants to comply with primary disinfection requirements. When applied to disinfection of water mains, however, two key differences should be noted. First, the bulk water used to fill the water main and inject ozone during the disinfection process is fully treated, finished water (typically with a chlorine residual) and does not require further disinfection. Second, the pipe wall harbors microbial contaminants and is the prime target of the disinfection process. But because CT exposure times are short and ozone is too reactive for effective penetration of ozone residuals into sediment or thick biofilms, effective cleaning of the pipe walls should be performed before ozonation in order for the ozonation process to be effective. Accordingly, pressure washing and flushing of sediments from the pipe should precede the disinfection process in order to avoid positive coliform failures and the need to repeat the disinfection procedure. The disinfection process should effectively treat organisms potentially attached to the stationary pipe wall as well as those that slough off into the flowing water.

Accordingly, the object of the treatment of water mains or other liquid-carrying conduits is to expose the walls of the pipeline to accumulating CT products for inactivation of the target organism or surrogate of interest. The effectiveness of ozone-based disinfection is dependent upon the CT value of the treating solution at the outlet of the pipeline section being treated.

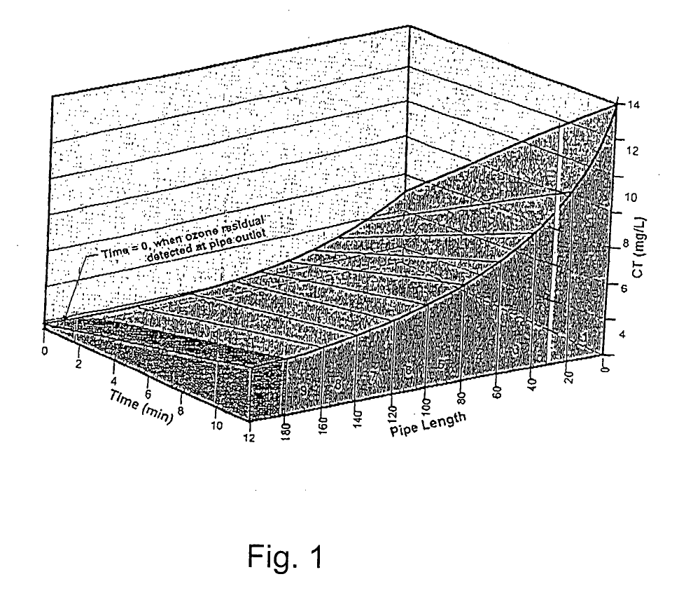

Referring to the drawings, and particularly to FIG. 1 thereof, there is shown a hypothetical surface plot of CT accumulation rates on pipeline walls for flow-through disinfection of a 200-ft long water main. As shown, the main is divided into ten 20-ft segments to explain the disinfection method. As ozonated water flows through the water main over time, the microbial contaminants attached to the pipeline are exposed to accumulating CT products over time, with the highest values occurring in upstream segment 1 and the lowest values in downstream segment 10. For any segment along the length of the pipeline, the CT product will increase in linear proportion to the exposure time. The CT value of the treating solution at the last segment of the pipeline, downstream segment 10, is monitored to determine whether the disinfection goal has been met, because that segment is exposed to the lowest accumulated CT product. The pipeline segments upstream of segment 10 will each accumulate significantly higher CT products than segment 10, thereby providing even greater inactivation rates of microbial contaminants within those segments. In operation, ozonated water must continue to flow through segment 10 until the accumulated CT product for segment 10 (i.e., the measured ozone residual concentration multiplied by the disinfection time) is greater than the required CT product for inactivation of the target organism.

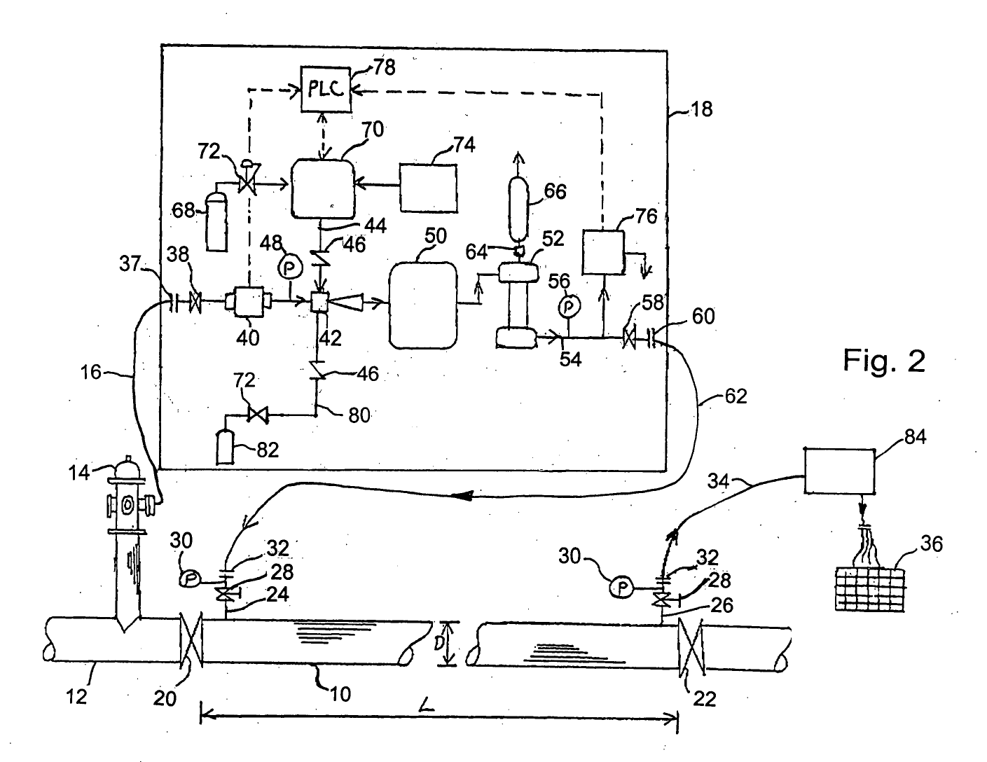

One embodiment of apparatus that can be employed to carry out ozone-based disinfection of pipelines and conduits is shown in FIG. 2. A pipeline segment 10 forming part of a water distribution system has an inner diameter D and a length L. Potable water is provided from a potable water supply pipeline 12 that includes a hydrant 14, or a similar flow takeoff connection, to allow potable water to flow into a conduit 16 that carries the potable water to an ozone treatment system 18.

Pipeline segment 10 is connected with water supply pipeline 12 at an inlet isolation valve 20. Spaced downstream from isolation valve 20 at distance L is an outlet isolation valve 22. Corporation taps 24, 26 are provided downstream of isolation valve 20 and upstream of isolation valve 22, respectively. Tap 24 is an inlet tap that allows the entry into pipeline segment 10 of treating solution containing ozone, and tap 26 is an outlet tap that allows the flow from pipeline segment 10 of the treating solution after it has passed through the interior of pipeline segment 10 from inlet tap 24. Each of taps 24, 26 includes a respective isolation valve 28, a pressure gauge 30 for monitoring the treating solution pressure entering and leaving pipeline segment 10, and a suitable connector 32, which can be a quick-connect quick-disconnect type of fitting. Connector 32 at the upstream end of pipeline segment 10 allows connection of the pipeline segment with ozone treatment system 18. Additionally, connector 32 at outlet tap 26 allows connection of the pipeline segment with an outlet conduit 34 to carry the treating solution that exits from pipeline segment 10 to a storm drain 36, a sewer, or to some other suitable disposal site.

Ozone treatment system 18 serves to generate ozone and to introduce the ozone into the potable water from supply pipeline 12 to provide the treating solution in the form of ozonated water. A water inlet connection 37 is followed by an isolation valve 38 that communicates with an inlet flow meter 40 to measure the rate of water flow into the ozone treatment system. Typical flow rates through such an ozone treatment system can range from about 150 gpm to about 200 gpm. Flow meter 40 is in communication with a venturi injector 42, in which ozone gas is introduced into the water flow stream under negative pressure through an ozone conduit 44 that includes a check valve 46 to prevent backflow of water into the source of ozone. A pressure gauge 48 is provided between flow meter 40 and venturi injector 42 to allow monitoring of the water pressure upstream of the injector.

Within venturi injector 42 the ozone and water mix under aggressive hydrodynamic conditions. The hydrodynamic mixing coupled with a nearly instantaneous water pressure change from positive pressure to negative pressure across the injector throat promotes highly efficient mass transfer of ozone into the water. A reaction vessel 50 is provided downstream of venturi injector 42 to reduce the water velocity and to provide a delay time for additional ozone gas/water contact under pressure to further enhance the mass transfer of the ozone gas into the water.

Downstream of reaction vessel 50 is a degassing separator 52 for removing unwanted entrained and stripped gases, primarily oxygen and ozone. The separator operation can be based upon a centrifugal process in which the ozone/water solution is introduced into the separator tangentially and accelerates to a velocity that exerts on it from about 4 to about 10 times the force of gravity, in the form of a lateral force, thereby creating a water film on the separator inner wall surface and a gas vortex at a central gas extraction core of the separator.

The water exits from separator 52 through a conduit 54 that includes a pressure gauge 56, and then flows through an isolation valve 58 to a connector 60. A conduit 62 extends from connector 60 to allow the ozonated treating solution to flow through conduit 62 to inlet connection 32 on pipeline segment 10. The separated gases accumulate at the top of separator 52 and exit through an air relief valve 64 into an offgas treatment cylinder 66, or the like, in which the gases are suitably treated before they are discharged into the atmosphere. Typically, about 98% of entrained gases can be removed from the water when using such a separator, thereby avoiding the buildup of gas pockets that could otherwise occur within the pipeline segment to be disinfected.

A venturi injector and downstream degassing separator for providing a liquid including a dissolved, liquid-soluble gas is disclosed in U.S. Pat. No. 5,674,312, entitled “Injection of Soluble Gas in a Liquid Stream and Removal of Residual Undissolved Gas,” which issued on Oct. 7, 1997, to Angelo L. Mazzei.

The ozone for disinfection can be produced from oxygen feed gas that is introduced into an ozone generator. The oxygen feed gas can be generated on site, such as by an oxygen pressure swing adsorption process that can deliver an oxygen flow rate of from about 80 scfh to about 160 scfh, or it can be provided in pressurized liquid oxygen cylinders. The ozone generation system can be relatively small and as such it can be a mobile system that can readily be mounted on a truck or trailer for portability. The ozone generation system, which is an air-cooled system, generates ozone from oxygen, it injects the ozone into a pressurized water flow stream, and it delivers the ozonated water into the pipeline segment to be treated. In the embodiment shown in FIG. 2, a liquid oxygen cylinder 68 is connected with an ozone generator 70 by a conduit that includes a pressure regulating valve 72 to meet the downstream operating pressure requirements of the ozone generator.

Ozone generator 70 can be an air- or water-cooled ozone generator that can produce ozone at an ozone-in-oxygen concentration ranging from about 6% to about 12% by weight. Depending upon the water flow rates to be used and the size of pipe segments to be disinfected, the capacity of the ozone generator can be of the order of from about 5 ppd to about 20 ppd. Because of the portability of the ozone generation system, the ozone generator preferably is air cooled and has ceramic tube or plate-type dielectrics to minimize breakage during use and transit, such as can be caused by vibration if transported by truck or trailer. However, the ozone generator can also be mounted on vibration isolation dampers, which facilitates the use of a truck- or trailer-mounted ozone system for use in field applications of pipeline disinfection.

The electrical power requirements for the ozone treatment system can range from about 1,200 watts to about 3,600 watts, depending upon the ozone production requirements and whether an on-site oxygen generation system is utilized. A portable, gasoline-engine-powered electrical power generator 74 of a readily available type and capacity can be utilized to supply the necessary electrical power requirements for operation of the system.

A programmable logic controller 78 is provided in the system for controlling system operation. Controller 78 is programmed to determine the required combination of initial ozone residual concentration and the decay rate constant of the ozonated water stream in order to maintain a target ozone residual of the order of from about 0.1 mg/L to about 0.2 mg/L at the outlet of the pipeline segment to be treated. Controller 78 is operatively connected with ozone generator 70 to automatically regulate the power input to the ozone generator to increase the ozone production rate to meet a particular CT product set point, based upon signals from an ozone analyzer 76, together with user-supplied information relating to the length and the inner diameter of the pipeline segment to be disinfected.

Ozone analyzer 76 serves to determine the initial ozone residual concentration and the ozone decay rate constant of the ozonated treating solution stream. The decay rate constant can be calculated by a microprocessor within the analyzer based upon measurements of the initial and final ozone residual concentrations over a predetermined time interval. The decay rate constant can be calculated based upon first order decay kinetics by the following equation:
K_{d}=ln(C/Cₒ)/T
where C is the final ozone residual in mg/L after a specified contact time, CO is the initial ozone residual in mg/L at the start of a specified contact time, T is the contact time in minutes, and K_{d} is the decay rate constant in min⁻¹.

One form of such an analyzer is disclosed in copending U.S. patent application Ser. No. 10/244,147, filed on Sep. 14, 2002, and entitled “Ozone-In-Water Decay Rate Analyzer,” naming Christopher R. Schulz as inventor, the entire disclosure of which is hereby incorporated herein by reference to the same extent as if fully rewritten.

As noted earlier, the ozone dose delivered to the pipeline segment to be treated should be sufficient to maintain an outlet ozone residual concentration of from about 0.2 mg/L to about 0.3 mg/L at the outlet of the pipeline segment to be treated. That residual level is sufficient to meet disinfection requirements, and it also is sufficiently low to allow ozonated water emanating from the pipeline segment being treated to be discharged to the environment without causing environmental harm. If higher residuals are used (>0.3 mg/L) an ozone neutralization device can be used to convert ozone into oxygen. Such a neutralization device can include a bucket-drip system, a tablet dispenser or tablets in a bag. Suitable ozone neutralization chemicals include ascorbic acid, sodium bisulfite, and calcium thiosulfate.

The accumulated CT product at the pipeline segment outlet increases with time as ozonated water discharges from the pipeline during the disinfection treatment time period, as is evident from the graph shown in FIG. 1. For example, a CT product target of 5 mg/L/min can be met by discharging ozonated water at a concentration of about 0.2 mg/L from the outlet end of the pipeline segment for a contact period of 25 minutes.

Also as noted earlier, disinfection requirements for water mains should be based upon meeting a temperature-dependent CT product for 4-log (or 99.99%) HPC inactivation. The CT product at the outlet end of the pipeline segment being treated should be capable of being met by maintaining an ozone residual of from about 0.2 mg/L to about 0.3 mg/L for the required time interval as water is discharged from the pipeline. For a given pipeline segment the outlet ozone residual can be predicted using the following equations:
C=(Cₒe^{-KdT})/T
and
   T=0.04(D²L/Q)
   where C is the outlet ozone residual concentration in mg/L,
Cₒ is the initial ozone residual concentration in mg/L,
K_{d} is the ozone decay rate constant in min⁻,
T is the contact time in minutes,
D is the pipeline inner diameter in inches,
L is the pipeline segment length in feet, and
Q is the water flow rate in gpm.

The equations given above are programmed into controller 78 and are part of the control logic used to automatically adjust ozone production rates to meet an outlet ozone residual concentration set point at the outlet of the pipeline segment. Controller 78 calculates the predicted outlet ozone residual concentration based upon the size of the pipeline segment and on-line measurements of water flow rate, initial ozone residual, and ozone decay rate constant. If the predicted value is less than the outlet ozone residual concentration set point at the outlet of the pipeline segment (typically about 0.5 mg/L to about 1 mg/L), controller 78 will automatically increase power to the ozone generator in predetermined increments until the predicted value and the set point value are within a predetermined difference range. Similarly, if the predicted value is greater than the set point value, controller 78 will automatically decrease power to the ozone generator in predetermined increments until those values are within a predetermined difference range.

Controller 78 can also be programmed to include a look-up table containing the CT product values for log inactivation of HPC bacteria at different water temperatures. A particular HPC log inactivation goal is entered (typically 2- to 4-log), along with a pipeline segment outlet ozone residual set point (typically from about 0.1 mg/L to about 0.2 mg/L), and a water temperature, and controller 78 will display the required end-of-pipeline contact time to be utilized for the disinfection process.

Also shown in FIG. 2 as a part of the ozone treatment system is a branch to enable the introduction into the pipeline treatment solution of carbon dioxide gas. In that regard, reducing the pH of the treatment solution operates to decrease the ozone decay rate, thereby enhancing the treatment efficacy by providing a higher ozone concentration within the pipeline being treated. The carbon dioxide concurrently increases the carbonate alkalinity of the treatment solution and reduces the pH, which advantageously can be maintained at a pH of about 6.0 or less. Specifically, a source 82 of carbon dioxide, which can be a standard pressurized carbon dioxide cylinder, provides a flow of carbon dioxide gas through conduit 80, in which the flow rate is regulated by pressure regulating valve 72, and through check valve 46 to venturi injector 42. Within venturi injector 42 the carbon dioxide, ozone, and water mix under aggressive hydrodynamic conditions. The hydrodynamic mixing coupled with a nearly instantaneous water pressure change from positive pressure to negative pressure across the injector throat promotes highly efficient mass transfer of ozone and carbon dioxide into the water.

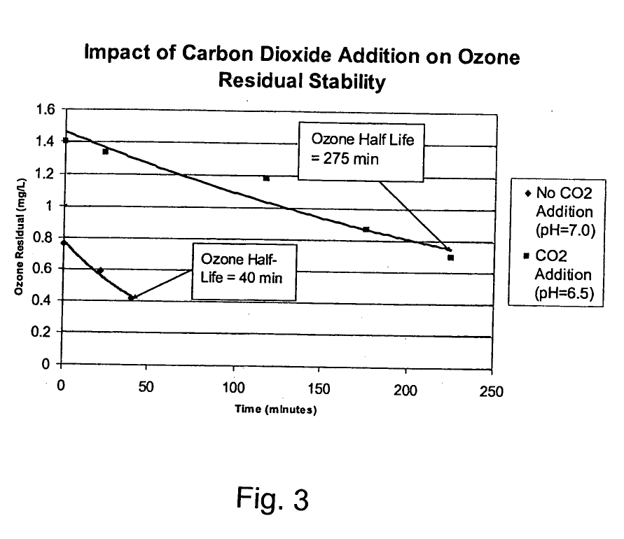

FIG. 3 shows the effect on ozone residual stability of the addition of carbon dioxide. Without the addition of carbon dioxide to reduce the pH of the supply water that has a pH of 7.0, the ozone half-life in the pipeline treatment solution is about 40 min. However, by adding sufficient carbon dioxide to reduce the pH of the pipeline treatment solution to about 6.5, the ozone half-life is extended to about 275 min, thereby providing improved disinfection by maintaining the pipeline surface exposed to a higher ozone residual than if no carbon dioxide were to be added.

In the operation of the embodiment shown in FIG. 2, pressurized water is provided from hydrant 14 to operate venturi injector 42 and to fill the pipeline segment with ozonated water. At that point carbon dioxide can be added through the venturi injector to lower the pH of the ozonated water, if the water introduced into the system has a relatively high pH. Typically, the water flow rate is from about 100 gpm to about 200 gpm. Generator 74 is started to supply electrical power to ozone generator 70, and oxygen gas is admitted to ozone generator 70 from liquid oxygen cylinder 68. The ozone flows from ozone generator 70 through conduit 44 and into venturi injector 42. Pressure regulating valve 72 is adjusted to maintain the desired oxygen gas flow rate for meeting the desired ozone production requirements. Ozone analyzer 76 is started and the power setting and ozone production rate are increased to provide an initial ozone residual concentration of about 0.5 mg/L, as measured by ozone analyzer 76. Preferably, the selected residual value is less than the required value for achieving disinfection objectives, so that excessive ozone residual concentrations do not occur at the pipeline segment outlet before optimization of the ozone production rate by the automated ozone control system. Ozone generator 70 is switched to automatic mode and controller 78 calculates the predicted outlet ozone residual concentration based upon the initial ozone residual concentration and the ozone decay rate constant measured by analyzer 76. The power provided to ozone generator 70 is then automatically increased to increase the ozone production rate to meet the set point outlet residual concentration. After the required disinfection contact time at the pipeline outlet has been reached the system can be shut down and the water remaining within the pipeline segment can be discharged to storm drain 36, or the like.

In order to neutralize the ozone residual, a neutralizer device 84 can be provided between the outlet connector 32 at the downstream end of the pipeline section being treated and the treatment solution discharge point, such as sewer drain 36, as shown in FIG. 2. Neutralizer device 84 can be a vessel or container into which a suitable chemical reducing agent is introduced to react with the residual ozone in the treatment solution. Examples of suitable chemical reducing agents include such compounds as sodium bisulfite, sodium metabisulfite, calcium thiosulfite, and ascorbic acid. In that regard, ascorbic acid and metabisulfites are readily available in tablet form and need only be replenished in neutralizer 84 when they have been almost consumed, in order to maintain a neutralizing function.

For relatively small pipeline lengths a single ozone injection point will be sufficient. For longer pipeline lengths or for large diameter pipelines, where higher ozone decay rates are more likely, multiple ozone injection points can be provided along the length of the pipeline in order to have overlapping ozone residual profiles to ensure that the entire pipeline length is adequately disinfected.

Although particular embodiments of the present invention have been illustrated and described, it will be apparent to those skilled in the art that changes and modifications can be made without departing from the spirit of the present invention. Accordingly, it is intended to encompass within the appended claims all such changes and modifications that fall with the scope of the present invention.

1. A method for disinfecting water supply pipelines, said method comprising:
a. providing a water supply pipeline to be disinfected, wherein the pipeline includes an inlet connection, and an outlet connection spaced along the pipeline from the inlet connection to define a predetermined pipeline length between the inlet connection and the outlet connection;
b. supplying pressurized water from a potable water source to an ozone treatment system that includes an ozone generator;
c. introducing pressurized gaseous oxygen into the ozone generator to provide gaseous ozone;
d. injecting the gaseous ozone into the pressurized water into a venturi injector within the ozone treatment system and at an ozone dose sufficient to maintain a predetermined ozone-in-water residual concentration at the pipeline outlet connection;
e. removing entrained gases from the ozonated pressurized water using a centrifugal degassing separator;
f. introducing the ozonated pressurized water from the venturi injector into the pipeline at the pipeline inlet connection;
g. maintaining a continuous flow of the ozonated pressurized water within the pipeline from the inlet connection to the outlet connection; and
h. regulating a discharge of ozonated pressurized water from the outlet connection to maintain a predetermined ozonated water pressure within the pipeline and a predetermined ozone-in-water residual concentration at the outlet connection for a time sufficient to meet a water supply disinfection requirement.

2. Apparatus for disinfecting a water supply pipeline, said apparatus comprising:
a. a source of gaseous ozone;
b. a venturi injector for introducing and mixing the gaseous ozone into pressurized potable water to provide an ozone-containing disinfectant solution having an ozone concentration sufficient to provide a predetermined ozone-in-water residual concentration at a pipeline outlet connection;
c. means for removing entrained gases from the ozone-containing disinfectant solution to minimize gas; pockets from forming in the pipeline to be disinfected;
d. means for introducing the disinfectant solution into a water supply pipeline at a pipeline inlet connection;
e. flow control means for regulating the rate of flow of the disinfectant solution within a pipeline to be treated to expose interior surfaces of the pipeline to the disinfectant liquid for a time sufficient to meet predetermined disinfection requirements;
f. wherein the apparatus is carried on a transportation vehicle for on-site disinfection of water supply pipelines at varying sites.

3. An ozone treatment system for introducing gaseous ozone into potable water under pressure to provide an ozone-containing disinfectant solution, said ozone treatment system comprising:
a. a source of ozone;
b. connection means for connection of the system with a source of pressurized potable water;
c. a venturi injector operatively coupled with the source of ozone and the source of pressurized potable water for introducing and mixing the ozone into the water;
d. an analyzer for measuring the rate of decay of an ozone residual concentration of the water;
e. a regulator for regulating the rate of ozone introduction into the water to provide a predetermined ozone-in-water concentration;
f. wherein the ozone treatment system is carried on a transportation vehicle.

4. A method in accordance with claim 1, wherein the ozone is provided from a source of oxygen.

5. A method in accordance with claim 1, wherein the flow of ozonated water is sufficient to substantially completely contact an inner wall surface of the pipeline.

6. A method in accordance with claim 1, wherein the predetermined ozone-in-water residual concentration is from about 0.2 mg/L to about 1 mg/L.

7. A method in accordance with claim 6, including the step of maintaining flow of the ozonated water within the pipeline for a time sufficient to provide in ozonated water at the pipeline outlet an ozone residual concentration such that a product of the ozone residual concentration and time of exposure of the pipeline to the ozonated water is from about 0.5 mg/L/min to about 5 mg/L/min.

8. A method in accordance with claim 1, including the step of analyzing the ozone concentration of the ozonated water before introduction of the ozonated water into the pipeline, and adjusting the potable water flow rate to maintain a desired ozone concentration in the ozonated water.

9. A method in accordance with claim 1, including the step of flushing the pipeline to dislodge and remove sediment from within the pipeline before treatment with the ozonated water.

10. Apparatus in accordance with claim 2, wherein the ozone source is an ozone generator that is supplied with one of liquid oxygen and gaseous oxygen.

11. Apparatus in accordance with claim 2, including an ozone analyzer between the means for introducing the ozone into the pressurized water and the pipeline.

12. Apparatus in accordance with claim 2, including a programmable logic controller operatively connected with an ozone generator for controlling an ozone production rate to provide a predetermined ozone concentration/time product in the disinfectant liquid at the pipeline outlet connection.

13. Apparatus in accordance with claim 12, wherein the programmable logic controller includes a lookup table of ozone concentration/time product values versus water temperature for controlling ozone concentration in the disinfectant liquid.

14. A method in accordance with claim 1, including the step of introducing gaseous carbon dioxide into the pressurized ozonated water to increase carbonate alkalinity and reduce pH of the water.

15. A method in accordance with claim 1, including the step of neutralizing an ozone residual of solution issuing from the pipeline outlet connection before disposal of the solution.

16. Apparatus in accordance with claim 2, including an ozone residual neutralization device positioned downstream of the pipeline outlet connection to minimize post-treatment off-gassing of ozone.

17. Apparatus in accordance with claim 2, including means for introducing gaseous carbon dioxide into the disinfectant solution before introduction of the solution into the pipeline.

18. Apparatus in accordance with claim 2, wherein the source of gaseous ozone is an ozone generator, and including vibration damping means for supporting the ozone generator to minimize damage to the ozone generator during transit of the apparatus.

19. A method in accordance with claim 1, including the step of spraying pressurized water containing ozone into the pipeline for initial interior flushing of the pipeline before disinfection by a continuous flow of ozonated water.
